(19) United States
(12) Patent Application Publication
(10) Pub. No.: US 2025/0268514 A1
(43) Pub. Date: Aug. 28, 2025

(54) LIDAR BASED MEDICAL DEVICE AND METHODS

(71) Applicant: LIDAVEX INC., Laguna Beach, CA (US)

(72) Inventor: Kamola MIRJALILOVA,, Laguna Beach, CA (US)

(21) Appl. No.: 19/064,608

(22) Filed: Feb. 26, 2025

Related U.S. Application Data
(60) Provisional application No. 63/559,172, filed on Feb. 28, 2024.

(51) Int. Cl. A61B 5/00; A61B 1/32

(52) U.S. Cl. CPC A61B 5/435; A61B 1/32; A61B 5/0062; A61B 5/742

(57) ABSTRACT

Described herein are light detection and ranging (LiDAR)-based medical devices and methods of their use for measuring internal body cavity geometries of a subject.

### RELATED DOCUMENTS

This application claims priority to U.S. Provisional Application No. 64/492,342, filed Feb. 28, 2024, the contents of which are herein incorporated by reference in their entirety.

### FIELD

This disclosure relates to a light detection and ranging (LiDAR)-based medical device and methods use.

### BACKGROUND

All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference. Citation or identification of any document in this application is not an admission that such document is available as prior art to the present invention.

During medical procedures it may be necessary to use medical devices to facilitate the viewing and measuring of human anatomy to evaluate a patient's wellness or disease status, and to provide medical providers with additional data with which they can diagnose conditions or diseases. For example, medical procedures related to women's wellness and pregnancy often involve the use of a speculum to aid in viewing a patient's cervix.

Cervical dilation measurements are an essential part of labor and delivery process to monitor the dynamics of labor. Measuring cervical dilation is also an essential step in third trimester routine check-ups to prevent unexpected pre-term labor. Accurate measurement of cervical dilation is essential for monitoring the progress of labor and determining appropriate interventions. Currently the standard method of performing cervimetry is a digital/manual method involving a specialist manually checking the degree of cervical dilation. Although manual cervimetry is the standard method in obstetric care, multiple problems are associated with this commonly used method, including patient pain and discomfort, provider subjectivity and variability, and risk of infections both for the mother and the fetus, which could be prevented by using a minimally invasive or noninvasive device. Further, manual assessments of cervical dilation can be subjective and prone to variability among healthcare providers because the measurements are based on how many fingers fit in the current cervical opening, with 1 finger considered as the consensus measurement of 1 cm, which is not accurate for every individual healthcare provider, i.e., one provider might have different thickness of fingers than another. These limitations can result in delayed or unnecessary interventions, including premature inductions or emergency deliveries, which carry risks for both mother and baby (Nixon, A. et al. (2015). Variability in cervical dilation assessments and labor management decisions. Obstetrics & Gynecology, 126(2), 235-240). Many female patients refuse routine cervimetry due to those factors, which increases health risks, including the risks of potential complications resulting from the lack of incomplete medical assessment.

Accurate cervical examinations are essential for effective obstetric care, particularly in monitoring labor progression and determining readiness for delivery. Inaccurate or delayed assessments of cervical dilation can lead to unnecessary medical interventions, such as cesarean sections or instrumental deliveries. Conversely, failure to recognize delayed cervical dilation can increase the risk of maternal and fetal complications, including birth trauma and asphyxia. Cervical dilation measuring devices provide objective and standardized measurements, improving the accuracy of assessment and contribute to patient safety by facilitating timely and accurate assessments thereby reducing the likelihood of unnecessary interventions or complications.

Certain medical devices are also used in non-obstetric care, such as otoscopes, laryngoscopes, laparoscopes and more. Many of these devices do not take individual patient introitusi into account and can cause patient pain and discomfort. Providing options of tools and instrumentation (e.g., devices) can minimize the invasiveness of particular medical procedures.

SUMMARY

This disclosure provides for an easily usable non-invasive medical device for medical professionals that provides an accurate, low-cost, easy-to-use approach to obtaining anatomical measurements and performing certain medical procedures. For example, the device facilitates non-invasive and accurate repeated cervical examination. The present invention uses a light detection and ranging (LiDAR) system, which provides a 2D or 3D spatial mapping of patient anatomies, including the vaginal cavity and cervix, for further analysis and accurate measurements. In certain embodiments, the laser used in the LiDAR system is a low-energy laser at wavelengths sufficient to obtain signals from biological tissue within a patient introitusi. Use of a low-energy laser reduces or excludes risks for the mother and the fetus when the device is used with pregnant subjects.

In some embodiments, the present invention provides a method of quick and painless cervimetry without any discomfort for the patient. Use of the LiDAR system in the device helps to identify certain measurements and qualitative assessments including cervical effacements and tissue softness by analyzing an intensity of the returning signal and backscattering characteristics of the laser pulses. Leveraging LiDAR technology in the medical device in cervimetry provides high-resolution point cloud maps of the cervix, enabling precise measurements of cervical dilation. This technological advancement has the potential to revolutionize obstetric diagnostics, offering more accurate and objective assessments for labor management.

In some embodiments, the medical device is a portable, handheld device operating on batteries, which makes it easily transportable and convenient to use by healthcare providers.

In some embodiments, portions of the device may function as a speculum if necessary. Due to the variability between individuals and variety of sizes of introitusi, some examinations and procedures might require more access for the LiDAR sensors than others, thus speculum would be used in those individuals to widen the opening and to allow for the laser pulses to access anatomies such as the cervix for accurate scanning. Moreover, in some embodiments said speculum comprise a rigid, non-bendable, body such that the operator of speculum can directly associate a particular directionality of the viewed direction with a particular aspect of a subject's introitusi. This is important when said introitusi are subject to movement because of a subject's movement, or the movement of a fetus during pregnancy which can alter the configuration of a mother's vaginal introitus (e.g., from a fetus kicking while within the womb).

In some embodiments, portions of the device are made of certain medical-grade materials, including polyurethane, polyphenylsulfone, heat-stabilized polypropylene, polyetheretherketone, polyvinyl chloride, acetal copolymer, ultra-high molecular weight polyethylene, or a combination thereof. In some embodiments, portions of the device are made of polyurethane (PU) for relative softness and stable temperature for the patient's comfort.

In some embodiments, portions of the device, in particular, portions of the tip of the device may have an opening with a diameter in a range from about 0.75 cm to about 9 cm. In some embodiments, the opening has a diameter that is about 0.50 to 0.75 cm, about 0.75 to 1 cm, about 1 to 2 cm, about 2 to 3 cm, about 3 to 4 cm, about 4 to 5 cm, about 5 to 6 cm, about 6 to 7 cm, about 7 to 8 cm, about 8 to 9 cm, about 9 to 9.5 cm, or ranging between any two diameters referred to above or herein.

In some embodiments, the medical device comprises: a body coupled to a handle; a tip comprising a plurality of petals defining an opening positioned on a distal end of the body of the medical device; and a LiDAR system comprising a low-level energy laser positioned within the body and proximate the tip; wherein the laser is positioned to align with the opening defined by the petals. In some embodiments, the body is non-deformable. The non-deformable body can be rigid. The rigid non-deformable body can be configured by a user to be directed to a selected region within a subject's introitusi to ensure the user is scanning said selected region.

In some embodiments, the opening on the distal end of the body of the medical device has a diameter in a range from about 0.75 cm to about 9 cm. In some embodiments, the opening has a diameter that is about 0.50 to 0.75 cm, about 0.75 to 1 cm, about 1 to 2 cm, about 2 to 3 cm, about 3 to 4 cm, about 4 to 5 cm, about 5 to 6 cm, about 6 to 7 cm, about 7 to 8 cm, about 8 to 9 cm, about 9 to 9.5 cm, or ranging between any two diameters referred to above or herein.

In some embodiments, the medical device further comprises an adjustment element that controls movement of at least some of the plurality of the petals during use.

In some embodiments, the medical device further comprises an adjustment element that controls movement of at least some of the plurality of the petals such that a user determines a position of the petals during use.

In some embodiments, the medical device further comprises an adjustment element that controls movement of at least some of the plurality of the petals such that the opening diameter is in a range from 1 cm to 7 cm during use. In some embodiments, the opening has a diameter that is about 0.50 to 0.75 cm, about 0.75 to 1 cm, about 1 to 2 cm, about 2 to 3 cm, about 3 to 4 cm, about 4 to 5 cm, about 5 to 6 cm, about 6 to 7 cm, about 7 to 8 cm, about 8 to 9 cm, about 9 to 9.5 cm, or ranging between any two diameters referred to above or herein during use.

In some embodiments, the laser of the medical device is fixedly positioned proximate the opening in the tip during use.

In some embodiments, the LiDAR system of the medical device comprises at least one of static LiDAR, near-field LiDAR, micropulse LiDAR, photon counting LiDAR, hybrid solid-state type LiDAR, flooded light array type (FLASH) LiDAR, time-of-flight (ToF) LiDAR, scanning LiDAR, solid-state LiDAR, or a sequence thereof, or combination thereof.

In some embodiments, the low-energy laser of the medical device emits light at a wavelength in a range from 400 nanometers to 1000 nanometers. In some embodiments, the light is at a wavelength of about 350 to 400 nm, about 400 to 450 nm, about 450 to 500 nm, about 500 to 550 degrees, about 550 to 600 degrees, about 650 to 700 nm, about 700 to 750 nm, about 750 to 800 nm, about 800 to 850 nm, about 850 to 950 nm, about 950 to 1000 nm, about 1000 to 1050 nm, or any ranging between any two wavelengths referred to above or herein.

In some embodiments, the tip comprises at least three petals.

In some embodiments, the tip comprises at least five petals.

In some embodiments, the tip comprises a plurality of petals comprised of at least one of polyurethane, polyphenylsulfone, heat-stabilized polypropylene, polyetheretherketone (PEEK), polyvinyl chloride, acetal copolymer, ultra-high molecular weight polyethylene, or a combination thereof.

In some embodiments, the medical device further comprises a processor and a display screen.

In some embodiments, the medical device comprises: a body comprising a light detection and ranging (LiDAR) system positioned within the body comprising a low-level energy laser; a speculum coupled to the body, comprising: a conical tip having a plurality of petals defining an opening positioned on a distal end of the body of the medical device; and an adjustment element that controls a size of the opening by movement of at least some of the plurality of the petals during use; wherein the laser is fixedly positioned within the body to align with the opening defined by the petals.

In some embodiments, the opening on the distal end of the body has a diameter in a range from 1 cm to 7 cm during use. In some embodiments, the opening has a diameter that is about 0.50 to 0.75 cm, about 0.75 to 1 cm, about 1 to 2 cm, about 2 to 3 cm, about 3 to 4 cm, about 4 to 5 cm, about 5 to 6 cm, about 6 to 7 cm, about 7 to 8 cm, about 8 to 9 cm, about 9 to 9.5 cm, or ranging between any two diameters referred to above or herein during use. In some embodiments, the opening has a diameter that is about 0.50 cm to 0.75 cm during use. In some embodiments, the opening has a diameter that is about 0.75 cm to 1 cm during use. In some embodiments, the opening has a diameter that is about 1 cm to 2 cm during use. In some embodiments, the opening has a diameter that is about 2 cm to 3 cm during use. In some embodiments, the opening has a diameter that is about 3 cm to 4 cm during use. In some embodiments, the opening has a diameter that is about 4 cm to 5 cm during use. In some embodiments, the opening has a diameter that is about 5 cm to 6 cm during use. In some embodiments, the opening has a diameter that is about 6 cm to 7 cm during use. In some embodiments, the opening has a diameter that is about 7 cm to 8 cm during use. In some embodiments, the opening has a diameter that is about 8 cm to 9 cm during use. In some embodiments, the opening has a diameter that is about 9 cm to 9.5 cm during use.

In some embodiments, the plurality of petals of the tip are arranged such that the conical tip is in an insertion configuration when the diameter of the opening is less than about 2 cm. In some embodiments, the insertion configuration is when the opening has a diameter than ranges from between about 0.75 cm to 2 cm. In some embodiments, the opening has a diameter that is about 0.50 to 0.75 cm, about 0.75 to 1 cm, about 1 to 2 cm, about 2 to 3 cm, or ranging between any two diameters referred to above or herein for insertion.

In one aspect, the present invention provides a method for providing a spatial mapping of an anatomy comprising: providing a medical device comprising: a speculum comprising: a plurality of polymer petals; a light detection and ranging (LiDAR) system comprising: a low-level energy laser emitting electromagnetic radiation comprising at least infrared radiation, visible radiation, invisible infrared radiation, near infrared radiation, or a combination thereof; inserting the speculum into an orifice of a subject; and using the speculum to dilate the orifice of the subject; using the LiDAR system to generate the spatial mapping of an anatomy; capturing the spatial mapping; analyzing the spatial mapping; and displaying the spatial mapping.

In some embodiments, the method further comprises identifying at least a feature of an anatomy comprising a measurement, position, consistency, vascularization, effacement, tissue softening, fetal station, fetal measurement, or a combination thereof; and capturing, analyzing, and displaying the feature.

In some embodiments, the method further comprises identifying a cervical effacement and tissue softness, capturing features of cervical effacement and tissue softness, and analyzing and displaying the features of cervical effacement and tissue softness.

In some embodiments the method further comprises identifying a region of rigid or damaged tissue, capturing features of the region of rigid or damaged tissue, and analyzing and displaying the features of the region of rigid or damaged tissue.

In some embodiments, the speculum has an opening defined by the petals having a diameter of less than 2 cm during insertion; and the opening has a diameter in a range from about 2 cm to about 7 cm during dilation of the orifice of the subject. In some embodiments, the opening has a diameter than ranges from between about 0.75 cm to 2 cm during insertion. In some embodiments, the opening has a diameter that is about 0.50 to 0.75 cm, about 0.75 to 1 cm, about 1 to 2 cm, about 2 to 3 cm or ranging between any two diameters referred to above or herein for insertion. In some embodiments, the opening has a diameter that ranges from between about 2 cm to about 7 cm during use as a speculum or dilator. In some embodiments, the opening has a diameter that is about 1 to 2 cm, 2 to 3 cm, about 3 to 4 cm, about 4 to 5 cm, about 5 to 6 cm, about 6 to 7 cm, about 7 to 8 cm, or ranging between any two diameters referred to above or herein during use.

In some embodiments, the opening has a diameter that ranges from between about 0.75 cm to 9 cm. In some embodiments, the diameter width varies from 1 cm to 7 cm depending on the individual characteristics of a patient. In some embodiments, the opening has a diameter that is about 0.50 to 0.75 cm, about 0.75 to 1 cm, about 1 to 2 cm, about 2 to 3 cm, about 3 to 4 cm, about 4 to 5 cm, about 5 to 6 cm, about 6 to 7 cm, about 7 to 8 cm, about 8 to 9 cm, about 9 to 9.5 cm, or ranging between any two diameters referred to above or herein. In some embodiments, the opening has a diameter that is about 0.50 cm to 0.75 cm. In some embodiments, the opening has a diameter that is about 0.75 cm to 1 cm. In some embodiments, the opening has a diameter that is about 1 cm to 2 cm. In some embodiments, the opening has a diameter that is about 2 cm to 3 cm. In some embodiments, the opening has a diameter that is about 3 cm to 4 cm. In some embodiments, the opening has a diameter that is about 4 cm to 5 cm. In some embodiments, the opening has a diameter that is about 5 cm to 6 cm. In some embodiments, the opening has a diameter that is about 6 cm to 7 cm. In some embodiments, the opening has a diameter that is about 7 cm to 8 cm. In some embodiments, the opening has a diameter that is about 8 cm to 9 cm. In some embodiments, the opening has a diameter that is about 9 cm to 9.5 cm.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the described invention solely to the specific embodiments described, may best be understood in conjunction with the accompanying drawings.

FIG. 1 illustrates a side view of a medical device having a main body (100), a tip (102) having a distal end diameter (110), an adjusting knob (104), a handle (108), and a start/stop button (112).

FIG. 2 illustrates a perspective view of a medical device having a main body (100),

a tip (102), and an adjusting knob (104). In some embodiments, the medical device has a display screen (106; not shown).

FIG. 3 illustrates a cross-sectional side view of a medical device showing the interior components of a main body (100) having a LiDAR system (114), an analysis processor (116), and a battery (118).

FIGS. 4A-F illustrate views and details of a tip (102). FIG. 4A illustrates a side view of a tip (102) having a distal end diameter (110). FIG. 4B illustrates a side view of a tip (102) having a distal end diameter (110) and an inner dilating component (103). FIG. 4C illustrates a front perspective view of a tip (102) with a dilated distal end diameter. FIG. 4D illustrates a back perspective view of a tip (102) with a dilated distal end diameter. FIG. 4E illustrates a side view of a tip (102) with a dilated distal end diameter. FIG. 4F illustrates a detailed view of two petals of a tip (102).

FIG. 5 illustrates a front view of the distal end of a tip (102).

FIG. 6 illustrates a side view of a medical device having a main body (100), a tip (102), an adjusting knob (104), a handle (108), and a start/stop button (112), where the tip (102) is positioned in a dilated configuration.

FIG. 7 illustrates a front view of the body of a medical device.

FIGS. 8A-D illustrate an inner component of a medical device. FIG. 8A illustrates a top view of an inner component of a medical device. FIG. 8B illustrates a bottom view of an inner component of a medical device. FIG. 8C illustrates a front view of the distal end of an inner component of a medical device. FIG. 8D illustrates a front view of the proximal end of an inner component of a medical device.

FIG. 9 illustrates a bottom view of the body of a medical device.

FIG. 10 illustrates a side view of a body (100) of a medical device.

FIG. 11 illustrates a front view of the distal end of a medical device.

FIG. 12 illustrates a side view of the medical device with the tip open.

FIG. 13 illustrates a side view of the medical device with the tip closed.

FIG. 14 illustrates an image obtained from using the medical device for cervimetry. Briefly, the tip (102) is positioned in a patient's orifice, e.g., a vaginal opening, putting into view a vaginal canal (128), a cervix (124), and a uterus (126), which low-energy laser pulses (130) are emitted towards, and laser backscattering (132) is returned to the medical device for analysis.

FIGS. 15A-15E shows images of the medical device used in a sheep model of cervimetry. FIG. 15A shows an image of an exemplary sheep reproductive organs. FIG. 15B shows an image of the uterine opening as viewed through an exemplary speculum. FIG. 15C shows a view of an exemplary medical device being used to map and measure a model of cervical opening. FIG. 15D shows a side view of an exemplary medical device being used to map and measure a model cervical opening. FIG. 15E is a picture of a model cervical opening being measured with medical tape.

FIGS. 16A-16C shows an example LiDAR point cloud map and information generated by the medical device. FIG. 16A shows an exemplary image of the LiDAR point cloud map. FIG. 16B shows an exemplary tabular readout of the angle, range, and intensity values corresponding to the point cloud map in FIG. 16A. FIG. 16C graphically illustrates the intensity vs counts, e.g., the reflected signal strength vs the number of detected points within the scan.

| Reference | # |
|---|---|
| main body/body | 100 |
| tip | 102 |
| inner dilating component | 103 |
| adjusting knob | 104 |
| display screen/screen | 106 |
| handle | 108 |
| distal end diameter | 110 |
| start/stop button | 112 |
| LiDAR system | 114 |
| analysis processor | 116 |
| battery | 118 |
| cervix | 124 |
| uterus | 126 |
| vaginal canal | 128 |
| low-energy laser pulses | 130 |
| laser backscattering | 132 |

### DETAILED DESCRIPTION

In the description, relative terms such as “lower,” “upper,” “horizontal,” “vertical,” “above,” “below,” “up,” “down,” “top,” and “bottom” as well as derivatives thereof (e.g., “horizontally,” “downwardly,” “upwardly,” etc.) should be construed to refer to the orientation as then described or as shown in the drawing under discussion. These relative terms are for convenience of description and do not require that the apparatus be constructed or operated in a particular orientation. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. The terms “couple,” “coupled,” “operatively coupled,” “operatively connected,” and the like should be broadly understood to refer to connecting devices or components together either mechanically, or otherwise, such that the connection allows the pertinent devices or components to operate with each other as intended by virtue of that relationship. The terms “fixedly,” “adjustably”, and he like should be construed to refer to the position as then described or shown in the drawing under discussion. These relative terms are for the convenience of description and do not require that the apparatus be constructed or operated in a particular position.

### A. Certain Exemplary Definitions

Before the present articles, devices, and/or methods are disclosed and described, it is to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. The terms “including,” “includes,” “having,” “has,” “with,” or variants thereof are used in either the detailed description and/or the claims, such terms are intended to be inclusive in a manner similar to the term “comprising.”

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as values between 10 and 15. For example, it is also understood that each unit between two particular units are also disclosed. For example, if 10 and 15 are disclosed, then 11, 12, 13, and 14 are also disclosed. In this application, if a data point range is disclosed, it is understood that each unit from the lowest data point to the highest stated datapoint, including the first (lowest) and last (highest) data point is disclosed. For example, if a data point range 1-20 is disclosed, it is understood that data points 1, 2, 3, 4, 5, 6, 7, 8, 9, 10, 11, 12, 13, 14, 15, 16, 17, 18, 19, and 20, and each unit between any two particular units in the range are also disclosed. It is also understood that whenever a series of values are disclosed, that any range falling between any two of the recited values is also understood to be included.

In this specification and in the claims which follow, reference will be made to a number of terms which shall be defined to have the following meanings:

“Optional” or “optionally” means that the subsequently described event or circumstance may or may not occur, and that the description includes embodiments where said event or circumstance occurs and embodiments where it does not.

The term “subject” refers to any individual who is the target of measurement. In some embodiments, the subject is a mammal. In some embodiments, the subject is human, non-human primate, bovine, equine, porcine, sheep, canine, or feline. In some embodiments, the subject is a zoo animal, for example, a panda, giraffe, or elephant. Thus, the subject can be a human or veterinary patient. The term subject, in some embodiments, refers to a “patient” under the treatment of a clinician, e.g., physician.

As used herein, the terms “pelvic exam,” “gynecological exam,” and “internal exam” broadly refer to a physical exam that checks the health of the female organs in the pelvis and includes cervical examination.

As used herein, “low-energy laser,” “cold laser,” and “low-level laser” refer to a Light Amplification by Stimulated Emission of Radiation (laser) that produces minimal heat and causes minimal temperature elevation. In some embodiments, the laser emits light at a wavelength in a range from between about 400 to 1000 nanometers. In some embodiments, the light is at a wavelength of about 350 to 400 nm, about 400 to 450 nm, about 450 to 500 nm, about 500 to 550 degrees, about 550 to 600 degrees, about 650 to 700 nm, about 700 to 750 nm, about 750 to 800 nm, about 800 to 850 nm, about 850 to 950 nm, about 950 to 1000 nm, about 1000 to 1050 nm, or any ranging between any two wavelengths referred to above or herein. Use of a low-energy laser reduces or excludes risks for the mother and the fetus when the device is used with pregnant subjects.

As used herein, the term “LiDAR” refers to light distance and ranging, which is a method of using a laser's reflection to determine the distance from a measured point. LiDAR is a remote sensing method that uses light in the form of a pulsed laser to measure ranges (variable distances) to a surface. These light pulses generate precise, three-dimensional information about the shape of the surface. A lidar instrument comprises a laser, a scanner, and a computer to associate a measured reflected laser signal to a selected position on a surface. Two most common types of lidar are topographic and bathymetric. Topographic lidar typically uses a near-infrared laser to map the dry surface, while bathymetric lidar uses water-penetrating green light to also measure surfaces in wet environments.

As used herein, light detection and ranging (LiDAR) systems can comprise a laser transmitter, receiver, scanner, and processing unit. In some embodiments, a LiDAR system utilizes pulsing laser beams into multiple locations to create a cloud map of a space and objects quickly and accurately, and a processor of the LiDAR system analyzes the returning signals with a software to provide a 2D or 3D spatial mapping and measurements. In some embodiments, a LiDAR system sends waves of light pulses or laser pulses in a spray of infrared dots or low-energy dots and measures each emission with its sensor to create a field of points that maps distances of objects in space then coalesces the dimensions and boundaries of a space and any objects in it. In some embodiments, the LiDAR system emits light using a laser to emit electromagnetic pulses then captures reflected light, creating detailed point cloud maps.

As used herein, the terms “tip,” “conical tip,” “speculum”, and “dilator” refer to a portion of the medical device comprising a plurality of petals defining an opening positioned on a distal end of the body of the medical device.

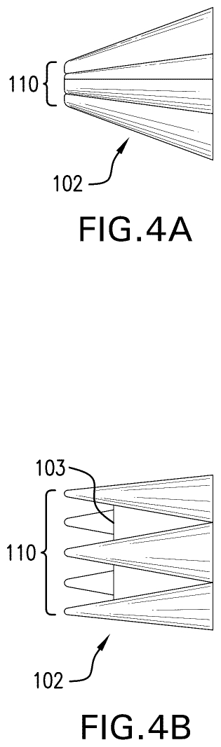

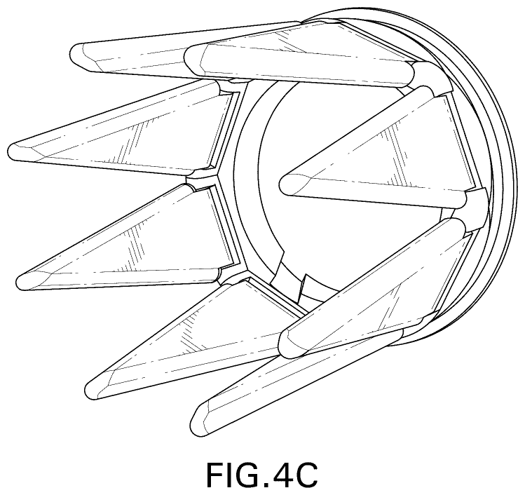

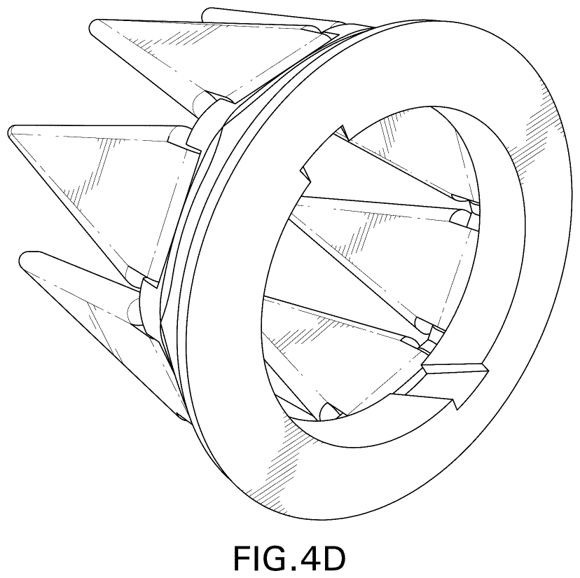

As used herein, the term “petal” refers to a component of the tip (102) of the medical device which rotates about a hinge as an inner dilating component (103) is rotated with an adjusting knob (104). FIG. 4F shows an exemplary illustration of two petals.

### B. Certain Exemplary Embodiments

This description of the exemplary embodiments is intended to be read in connection with the accompanying drawings, which are to be considered part of the entire written description. It should be understood, however, that the present disclosure is not intended to be limited to the particular forms disclosed and that the drawings are not necessarily shown to scale. Rather, the present disclosure covers all modifications, equivalents, and alternatives that fall within the spirit and scope of these exemplary embodiments.

The present disclosure includes embodiments of a LiDAR-based medical device that may be configured to examine certain anatomies of a patient during medical procedures, for example, during laparoscopic procedures or for cervimetry as part of a gynecological exam.

In some embodiments, the medical device has a detection range of between about 2 to 30 cm and an angular resolution of about 0.6 degrees, ensuring high precision in measurements. In some embodiments, the detection range is from about 2 to 30 cm, or any centimeter range falling between about 2 cm to about 30 cm. In some embodiments, the angular resolution is between about 0.2 to 1 degree, or any tenth of a degree range falling between about 0.2 and 1 degree. In some embodiments, the angular resolution is about 0.2 to 0.3 degrees, about 0.3 to 0.4 degrees, about 0.4 to 0.5 degrees, about 0.5 to 0.6 degrees, about 0.6 to 0.7 degrees, about 0.7 to 0.8 degrees, about 0.8 to 0.9 degrees, about 0.9 degrees to 1 degree, or any ranging between any two degrees referred to above or herein.

In some embodiments, the LiDAR system of the medical device comprises at least one of static LiDAR, near-field LiDAR, micropulse LiDAR, photon counting LiDAR, hybrid solid-state type LiDAR, flooded light array type (FLASH) LiDAR, time-of-flight (ToF) LiDAR, scanning LiDAR, solid-state LiDAR, or a sequence thereof, or combination thereof. In some embodiments, a LiDAR system is used to determine the rigidity of objects in a space. In some embodiments, a LiDAR system is used to provide 2D spatial mapping of patient anatomies, including the vaginal cavity and cervix, for further analysis and accurate measurements. In some embodiments, a LiDAR system is used to provide 3D spatial mapping of patient anatomies, including the vaginal cavity and cervix, for further analysis and accurate measurements. In some embodiments, solid-state LiDAR is used to provide 2D spatial mapping of patient anatomies, including the vaginal cavity and cervix, for further analysis and accurate measurements.

In some aspects, the present invention is an easily usable non-invasive medical device for medical professionals that provides an accurate, low-cost, easy-to-use approach to obtaining anatomical measurements and performing certain medical procedures. For example, the device facilitates non-invasive and accurate repeated cervical examination. As used herein, the terms “pelvic exam,” “gynecological exam,” and “internal exam” broadly refer to a physical exam that checks the health of the female organs in the pelvis and includes cervical examination.

In some embodiments, the present invention provides a method of quick and painless cervimetry without any discomfort for the patient. Use of the LiDAR system in the device helps to identify certain measurements and qualitative assessments including cervical effacements and tissue softness by analyzing an intensity of the returning signal and backscattering characteristics of the laser pulses.

A Light Amplification by Stimulated Emission of Radiation (laser) is used in embodiments of the LiDAR-based medical device. In some aspects, the laser used in the LiDAR system is known as a low-energy laser. As used herein, “low-energy laser,” “cold laser,” and “low-level laser” refer to a laser that produces minimal heat and causes minimal temperature elevation. In some embodiments, the laser emits light at a wavelength in a range from about 400 to 1000 nanometers. In some embodiments, the light is at a wavelength of about 350 to 400 nm, about 400 to 450 nm, about 450 to 500 nm, about 500 to 550 degrees, about 550 to 600 degrees, about 650 to 700 nm, about 700 to 750 nm, about 750 to 800 nm, about 800 to 850 nm, about 850 to 950 nm, about 950 to 1000 nm, about 1000 to 1050 nm, or any ranging between any two wavelengths referred to above or herein. Use of a low-energy laser reduces or excludes risks for the mother and the fetus when the device is used with pregnant subjects.

In some embodiments, the medical device is a portable, handheld device operating on batteries, which makes it easily transportable and convenient to use by healthcare providers. In some embodiments, the medical device operates on a rechargeable battery. In some embodiments, the medical device operates with a plugged-in power source.

In some embodiments, portions of the device may function as a speculum if necessary. As used herein, the terms “tip,” “conical tip,” “speculum”, and “dilator” refer to a portion of the medical device comprising a plurality of petals defining an opening positioned on a distal end of the body of the medical device. Due to the variability between individuals and variety of sizes of introitusi, some examinations and procedures might require more access for the LiDAR sensors than others, thus speculum would be used in those individuals to widen the opening and to allow for the laser pulses to access anatomies such as the cervix for accurate scanning.

As used herein, the term “petal” refers to a component of the tip of the medical device. In some embodiments, each petal of a tip (102) rotates about a hinge as an inner dilating component (103) is rotated with an adjusting knob (104). FIG. 4F shows an exemplary illustration of two petals.

In some embodiments, the medical device contains a LiDAR system positioned within the body and proximate the tip, wherein the laser is positioned to align with the opening defined by the petals. In some embodiments, the opening has a diameter that ranges from between about 0.75 cm to 9 cm. In some embodiments, the diameter width varies from 1 cm to 7 cm depending on the individual characteristics of a patient. In some embodiments, the opening has a diameter that is about 0.50 to 0.75 cm, about 0.75 to 1 cm, about 1 to 2 cm, about 2 to 3 cm, about 3 to 4 cm, about 4 to 5 cm, about 5 to 6 cm, about 6 to 7 cm, about 7 to 8 cm, about 8 to 9 cm, about 9 to 9.5 cm, or ranging between any two diameters referred to above or herein. In some embodiments, the opening has a diameter that is about 0.50 cm to 0.75 cm. In some embodiments, the opening has a diameter that is about 0.75 cm to 1 cm. In some embodiments, the opening has a diameter that is about 1 cm to 2 cm. In some embodiments, the opening has a diameter that is about 2 cm to 3 cm. In some embodiments, the opening has a diameter that is about 3 cm to 4 cm. In some embodiments, the opening has a diameter that is about 4 cm to 5 cm. In some embodiments, the opening has a diameter that is about 5 cm to 6 cm. In some embodiments, the opening has a diameter that is about 6 cm to 7 cm. In some embodiments, the opening has a diameter that is about 7 cm to 8 cm. In some embodiments, the opening has a diameter that is about 8 cm to 9 cm. In some embodiments, the opening has a diameter that is about 9 cm to 9.5 cm. In some embodiments, the plurality of petals of the tip are arranged such that the conical tip is in an insertion configuration when the diameter of the opening is less than about 2 cm. In some embodiments, the tip is customizable.

In some embodiments, the tip is decouplable from the body of the medical device, for example, if a tip is customized for a specific anatomical structure of a subject such as an ear.

As featured and disclosed herein, certain medical-grade materials include or exclude polyurethane, polyphenylsulfone, heat-stabilized polypropylene, polyetheretherketone, polyvinyl chloride, acetal copolymer, ultra-high molecular weight polyethylene, and similar materials. In some embodiments, portions of the device are made of certain medical-grade materials, including polyurethane, polyphenylsulfone, heat-stabilized polypropylene, polyetheretherketone (PEEK), polyvinyl chloride, acetal copolymer, ultra-high molecular weight polyethylene, or a combination thereof. In some embodiments, portions of the device are made of polyurethane (PU) for relative softness and stable temperature for the patient's comfort. In some embodiments, the tip is made of medical-grade material referred to above or herein. In some embodiments, the tip is made of PU.

In some embodiments, the tip comprises a plurality of petals. In some embodiments, the tip comprises at least two petals. In some embodiments, the tip comprises at least three petals. In some embodiments, the tip comprises at least four petals. In some embodiments, the tip comprises at least five petals. In some embodiments, the tip comprises more than five petals. In some embodiments, the tip is comprised of 5 individual petals and in a closed state it has a conical shape, the distal end still allowing the LiDAR signals to pass easily due to the diameter of about 1 cm.

In some embodiments, the medical device further comprises an adjustment element that controls movement of at least some of the plurality of the petals during use.

In some embodiments, the medical device further comprises an adjustment element that controls movement of at least some of the plurality of the petals such that a user determines a position of the petals during use.

In some embodiments, the medical device further comprises an adjustment element that controls movement of at least some of the plurality of the petals such that the distal end of the tip, i.e., the opening diameter, is in a range from 1 cm to 7 cm or ranging between any two diameters referred to above or herein during use. In some embodiments, the opening diameter is about 0.50 to 0.75 cm, about 0.75 to 1 cm, about 1 to 2 cm, about 2 to 3 cm, about 3 to 4 cm, about 4 to 5 cm, about 5 to 6 cm, about 6 to 7 cm, about 7 to 8 cm, about 8 to 9 cm, about 9 to 9.5 cm, or ranging between any two diameters referred to above or herein during use.

In some embodiments, the laser of the medical device is fixedly positioned proximate the opening in the tip during use.

In some embodiments, the medical device further comprises a processor (116) and a display screen (106). In some embodiments, the main analysis is performed on a processor which has a specifically designed software for cervimetry and entire cervical assessment that will be displayed on the screen, including, as non-limiting examples, intensity and backscattering characteristics analysis for effacement and softness. In some embodiments, the analysis processor identifies, from LiDAR 2D or 3D spatial map, an inner circumference of the cervix defining the cervical dilation from about 0 to 10 cm. In some embodiments, the processor computes the data on effacement on the scale of 0-100% and any percentage within that range, which would be identified from the intensity of the reflected pulses and their penetration rate. In some embodiments, the data on effacement is combined with analysis of backscattering light using the processor is used to assess the tissue softness and provide complex data on labor dynamics. In some embodiments, the medical device is optionally coupled to a processor and a display screen.

In some embodiments, the medical device further comprises a communication device for transmitting a message to a remote receiver, and the receiver connected to a computer system. and/or receiver for any type of wide-ranging wireless communication is conceivable.

In some embodiments, the medical device further comprises a power unit for powering the medical device. The power unit may comprise a battery. The power unit may comprise charging circuitry connected to the battery and terminals of a selected portion of the outer surface of the medical device for charging the battery. The power unit may comprise separate charging terminal(s) for charging the battery.

The medical device may comprise a sensor unit with one or more sensors. The sensor unit is connected to a processing unit within the medical device for feeding sensor data to the processing unit. The sensor unit may comprise an accelerometer for sensing acceleration and provision of acceleration data to the processing unit. The sensor unit may comprise a temperature sensor for provision of temperature data to the processing unit.

The medical device may comprise an interface connected to the processor. The interface may be configured as an accessory interface for connecting, e.g. wirelessly connecting, the medical device to one or more accessory devices. The interface may comprise an antenna and a wireless transceiver, e.g. configured for wireless communication at frequencies in the range from 2.4 to 2.5 GHZ. The wireless transceiver may be a Bluetooth transceiver, i.e. the wireless transceiver may be configured for wireless communication according to Bluetooth protocol, e.g. Bluetooth Low Energy, Bluetooth 4.0, Bluetooth 5. The interface optionally comprises a loudspeaker and/or a haptic feedback element for provision of an audio signal and/or haptic feedback to the user, respectively.

In some embodiments, the interface comprises a device for transmitting data provided from other equipment of the medical device to an external medium, particularly preferably the medical device. Expediently, an device is an interface according to the USB (Universal Serial Bus) standard and/or according to the Bluetooth Standard. The interface device is preferably arranged on the outer surface of the medical device.

In some embodiments, the medical device further comprises a wireless emitter and receiver. The wireless emitter and receiver can be selected from bluetooth, ZigBee, or WiFi. The wireless emitter and receiver transmit processed data to a central processing unit (CPU) configured for paired viewing. In some embodiments, the medical device is paired with an augmented reality visualizer.

In some aspects, the medical device is non-flexible and has a grip-and-pull model which helps to view the posterior cervix by gently pulling it for LIDAR access. Such a grip and pull model is achieved through the coating of the speculum tip that grips to the vaginal wall and rotational movement of the speculum that brings the cervix downwards. In contrast to a flexible inserter that requires active manipulation to be configured to an orifice such as a vaginal cavity's contouring, the present invention does not require further active manipulation to be correctly positioned for use such as in cervimetry. The present invention therefore advantageous over a medical device with a flexible inserter and is further advantageous in that patient comfort is maximized, and the potential for misalignment or misapplication of the device in an orifice is minimized.

In some embodiments, the medical device comprises: a body coupled to a handle; a tip comprising a plurality of petals defining an opening positioned on a distal end of the body of the medical device; and a LiDAR system comprising a low-level energy laser positioned within the body and proximate the tip; wherein the laser is positioned to align with the opening defined by the petals.

In some embodiments, the medical device comprises: a body comprising a LiDAR system positioned within the body comprising a low-level energy laser; a speculum coupled to the body, comprising: a conical tip having a plurality of petals defining an opening positioned on a distal end of the body of the medical device; and an adjustment element that controls a size of the opening by movement of at least some of the plurality of the petals during use; wherein the laser is fixedly positioned within the body to align with the opening defined by the petals.

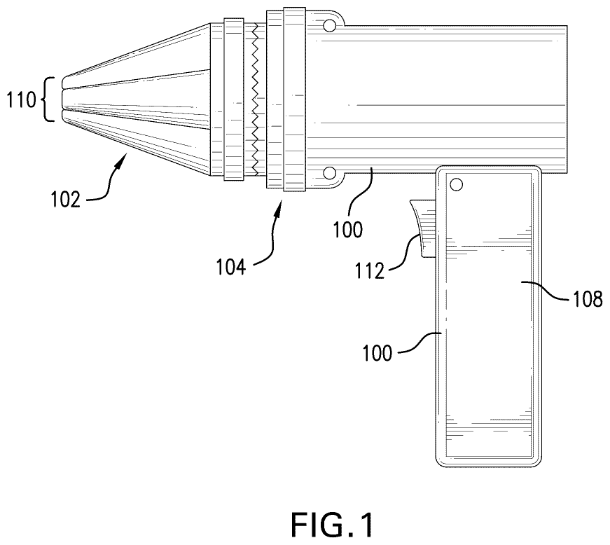

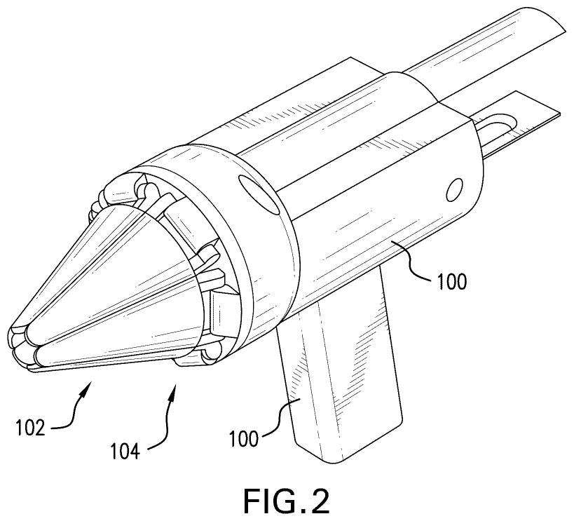

As shown in FIG. 1 and FIG. 2, examples of a medical device comprising a main body (100), a tip (102) having a distal end diameter (110), an adjusting knob (104), a handle (108), and a start/stop button (112).

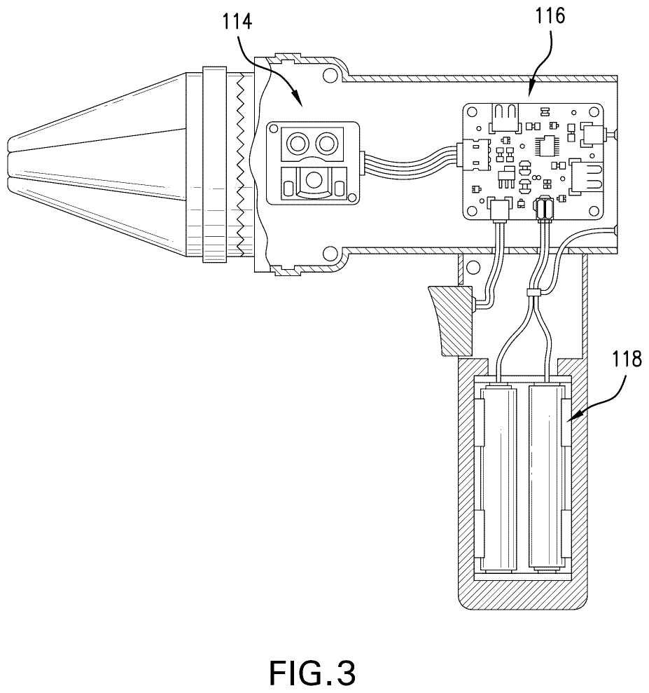

Exemplary aspects of the medical device include interior components of the main body (100), for example as illustrated in FIG. 3.

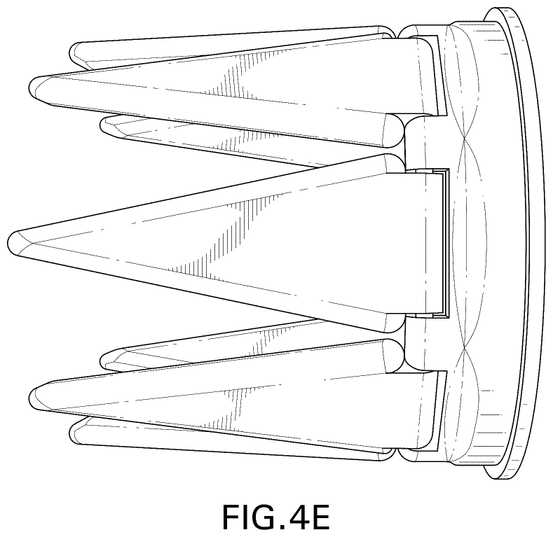

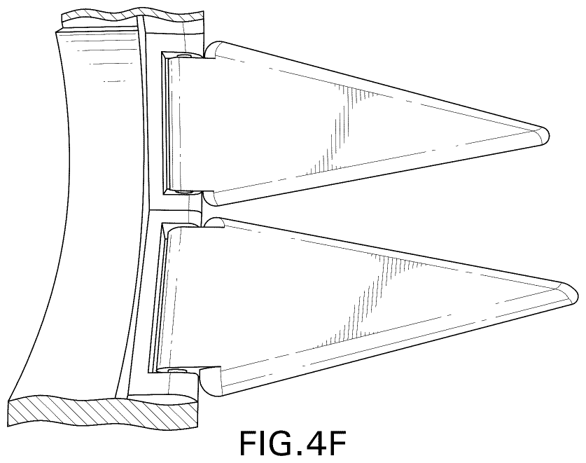

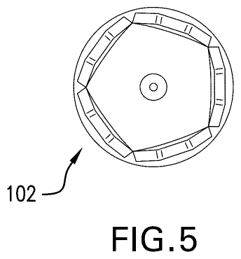

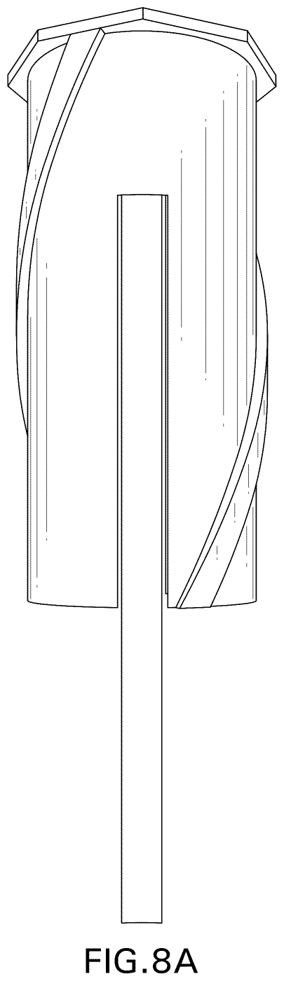

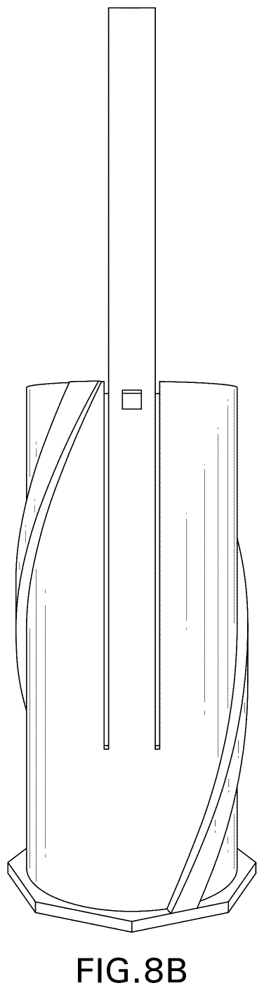

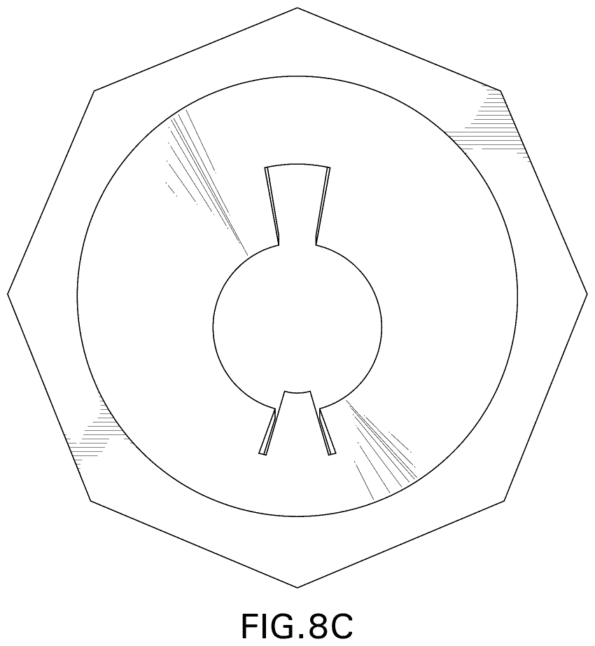

As shown in FIG. 4 and FIG. 5, an exemplary tip is used in a closed configuration or an open, e.g., dilated, configuration. An exemplary inner dilating component (103) controlling the relative position of the distal end diameter (110) of the tip is also exemplified in FIG. 8. Each petal of an exemplary tip (102) rotates about a hinge in response to an inner dilating component (103) being rotated as controlled by an adjusting knob (104). Rotation in one direction of the adjusting knob (104) controlling the rotation of an inner dilating component (103), e.g., clockwise, results in the controlled dilation of an exemplary tip, for example, to function as a speculum. Conversely, a counter-directional rotation of the adjusting knob (104) controlling the rotation of an inner dilating component (103), e.g., counterclockwise, results in the controlled closure of an exemplary tip.

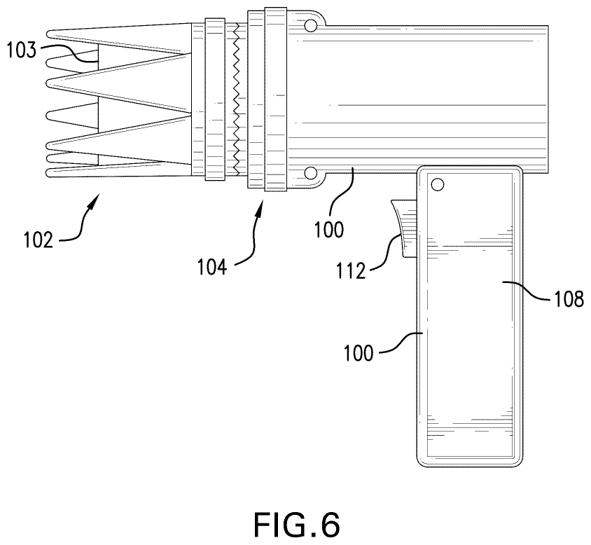

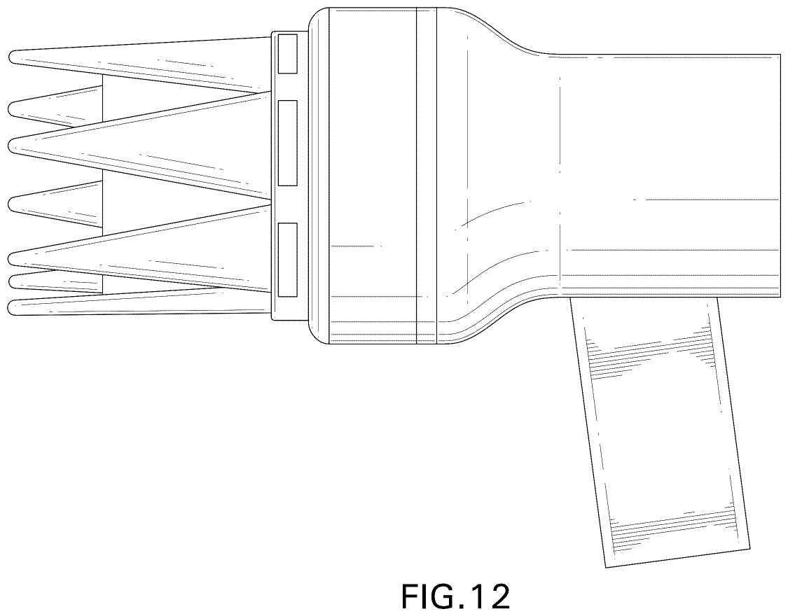

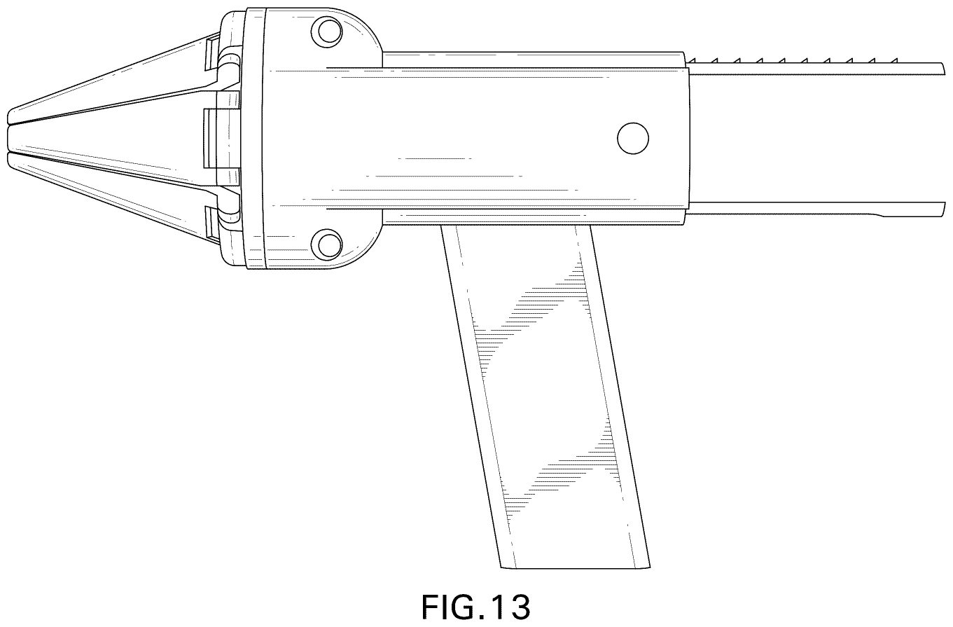

Examples of the medical device with the tip (102) in a dilated configuration is shown in FIG. 6 and FIG. 12. An example of the medical device with the tip (102) in a closed configuration is shown in FIG. 1, FIG. 2, FIG. 3, and FIG. 13.

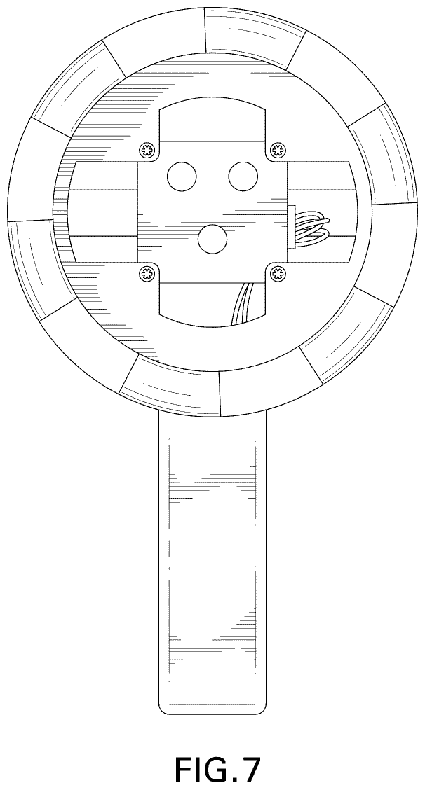

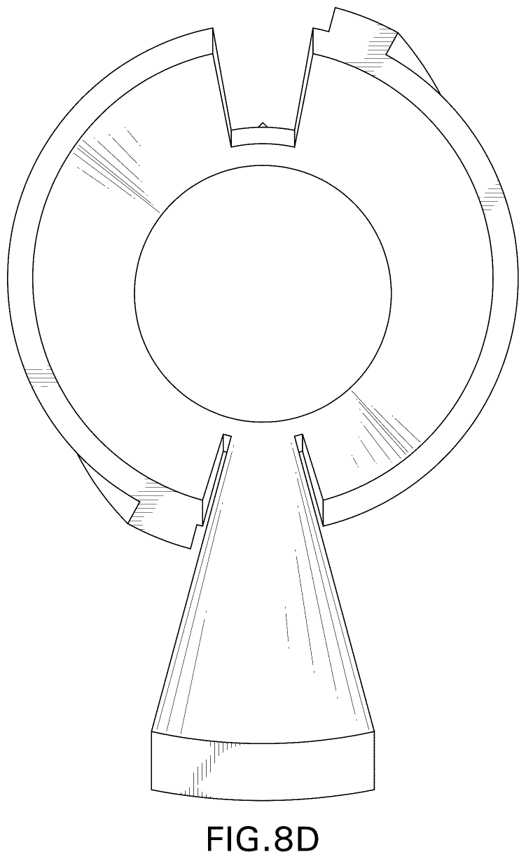

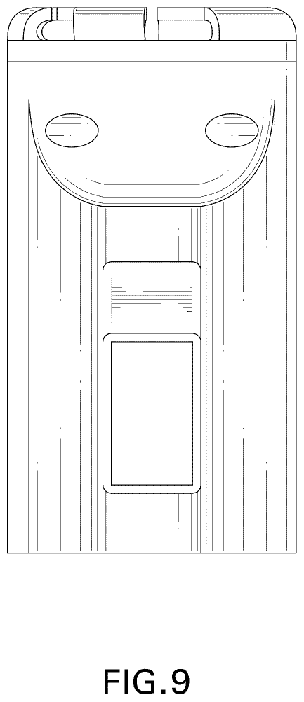

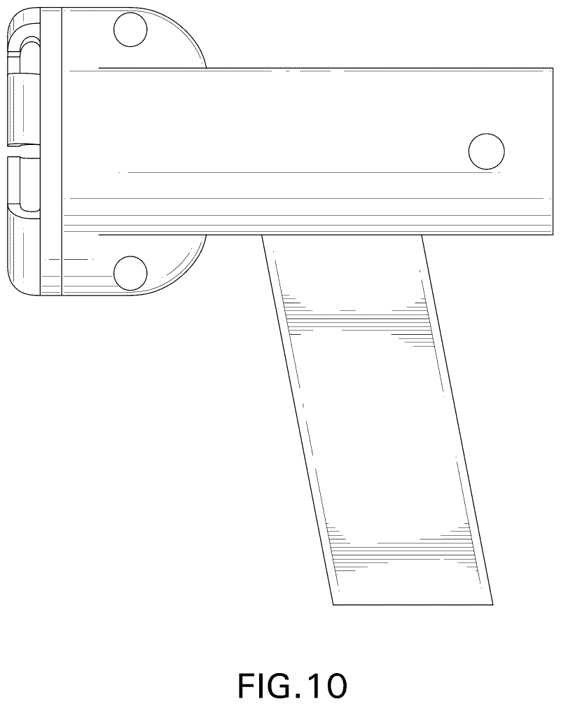

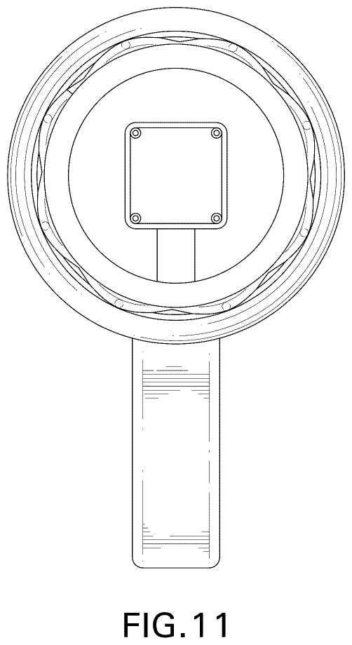

As shown in FIG. 7, FIG. 9, FIG. 10, and FIG. 11, in some embodiments the tip (102) is decouplable from the body (100) of the medical device, for example, if a tip (102) is customized for a specific anatomical structure of a subject.

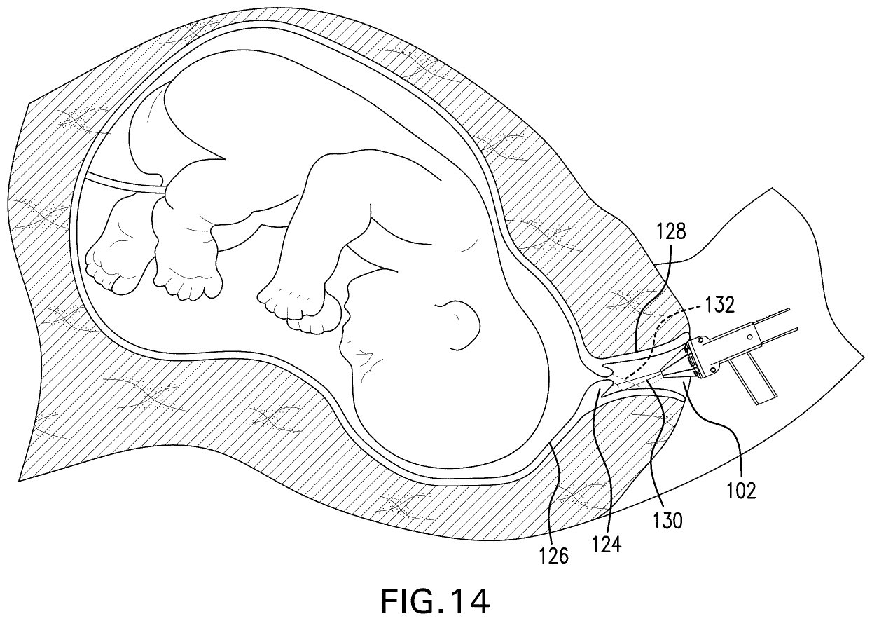

As a non-limiting example, FIG. 14 illustrates an image of using the medical device for cervimetry, in which the tip (102) is positioned in a patient's orifice, e.g., a vaginal opening, putting into view a vaginal canal (128), a cervix (124), and a uterus (126), which low-energy laser pulses (130) are emitted towards, and laser backscattering (132) is returned to the medical device for analysis.

### C. Certain Exemplary Methods

In one aspect, the present invention provides a method for providing a spatial mapping of an anatomy comprising: providing a medical device comprising: a speculum comprising: a plurality of polymer petals; a light detection and ranging (LiDAR) system comprising: a low-level energy laser emitting electromagnetic radiation comprising at least infrared radiation, visible radiation, invisible infrared radiation, near infrared radiation, or a combination thereof; inserting the speculum into an orifice of a subject; and using the speculum to dilate the orifice of the subject; using the LiDAR system to generate the spatial mapping of an anatomy; capturing the spatial mapping; analyzing the spatial mapping; and displaying the spatial mapping.

In some embodiments, the method further comprises identifying at least a feature of an anatomy comprising a measurement, position, consistency, vascularization, effacement, tissue softening, fetal station, fetal measurement, or a combination thereof; and capturing, analyzing, and displaying the feature.

In some embodiments, the method further comprises identifying a cervical effacement and tissue softness, capturing features of cervical effacement and tissue softness, and analyzing and displaying the features of cervical effacement and tissue softness.

In some embodiments the method further comprises identifying a region of rigid or damaged tissue, capturing features of the region of rigid or damaged tissue, and analyzing and displaying the features of the region of rigid or damaged tissue.

In some embodiments, the speculum has an opening defined by the petals having a diameter of less than 2 cm during insertion; and the opening has a diameter in a range from about 2 cm to about 7 cm during dilation of the orifice of the subject.

Although the embodiments of the present invention as provided in this disclosure have been described in detail, it should be understood that various changes, substitutions and alterations can be made herein without departing from the spirit and scope of the invention as defined in the appended claims.

### EXAMPLES

The specific methods and compositions described herein are representative of preferred embodiments and are exemplary and not intended as limitations on the scope of certain aspects of the invention. Other objects, aspects, and embodiments will occur to those skilled in the art upon consideration of this specification, and are encompassed within the spirit of the invention as defined by the scope of the claims. Thus, for example, in each instance herein, and in embodiments or examples of the present invention, any of the terms “comprising”, “consisting essentially of”, and “consisting of” may be replaced with either of the other two terms in the specification. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation agreed to and expressly adopted in a responsive writing by Applicants. Furthermore, titles, headings, or the like are provided to enhance the reader's comprehension of this document and should not be read as limiting the scope of the present invention. Any examples of aspects, embodiments, or components of the invention referred to herein are to be considered non-limiting.

### Example 1

### In Vivo Sheep Model

A proof-of-concept study using sheep reproductive organs, demonstrating the exemplary device's capability using LiDAR to capture precise point cloud maps and measure cervical dilation with high accuracy. The device demonstrated a mean absolute error (MAE) of 0.9 mm and a coefficient of variation (CV) of 3.2%, indicating its potential for accurate obstetric diagnostics, and the results suggest significant potential for the device to improve obstetric care, particularly in monitoring labor progression and ensuring timely interventions.

Sheep reproductive organs were selected for this study due to their anatomical and physiological similarities to human reproductive systems (Smith, L. et al. (2018). The use of animal models in reproductive health research. Reproductive Biology, 18(2), 97-104), making them a suitable model for preliminary testing of obstetric devices.

Materials and Methods

Ethical Considerations: Sheep reproductive organs were ethically sourced from abattoirs, adhering to research ethics and animal welfare standards (Doe, J., et al. (2019). Ethical sourcing of animal tissues for biomedical research. Bioethics Journal, 33(2), 85-96). Ethical approval was obtained from the Institutional Review Board (IRB) for the use of animal tissues in this study.

Device and Technology: The exemplary device employs LiDAR technology to emit laser pulses and capture reflected light, creating detailed point cloud maps. The device has a detection range of 2-30 cm and an angular resolution of 0.6 degrees, ensuring high precision in measurements.

### Procedure:

1. Preparation of Organs: Sheep reproductive organs were preserved following standardized protocols to maintain anatomical integrity (Brown, T. et al. (2016). Preservation of animal reproductive organs for biomedical research. Journal of Animal Science, 94(3), 123-132).

2. LiDAR Mapping: An exemplary LiDAR-based medical device was used to capture point cloud maps of the cervix, simulating conditions during labor.

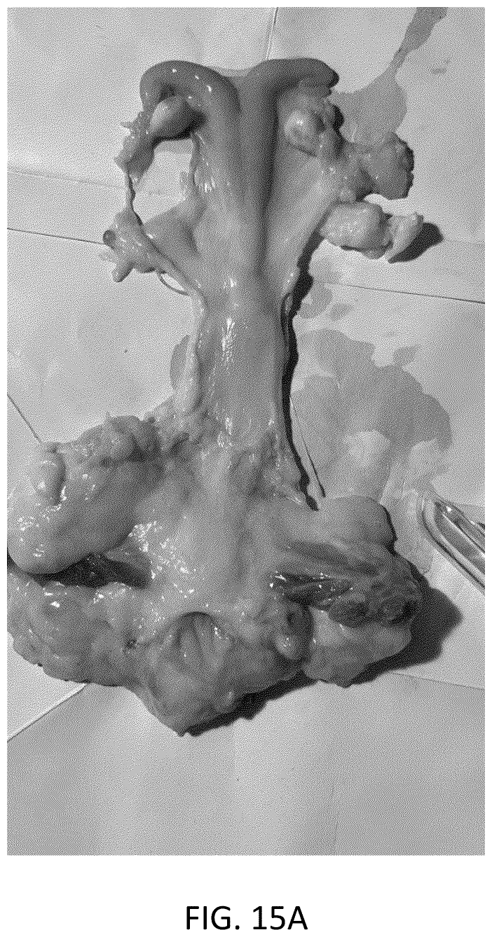

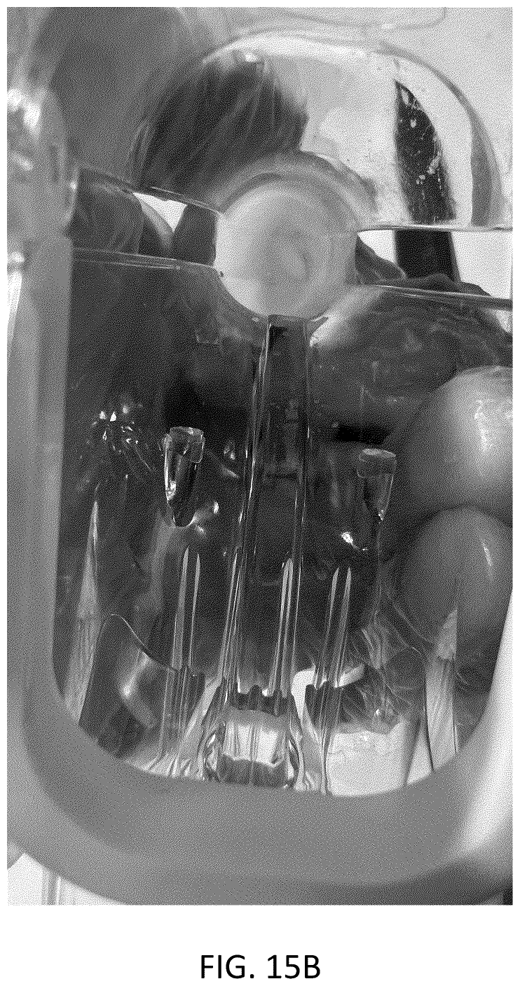

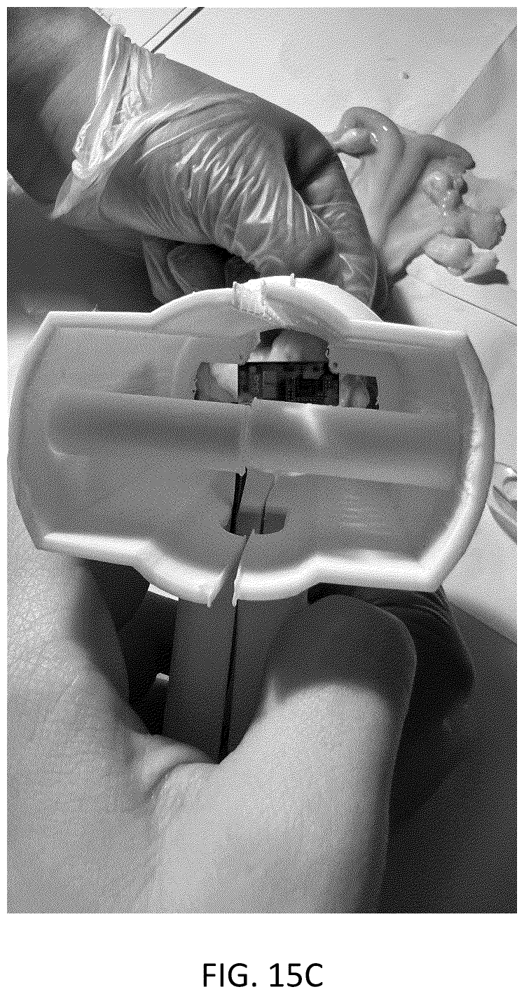

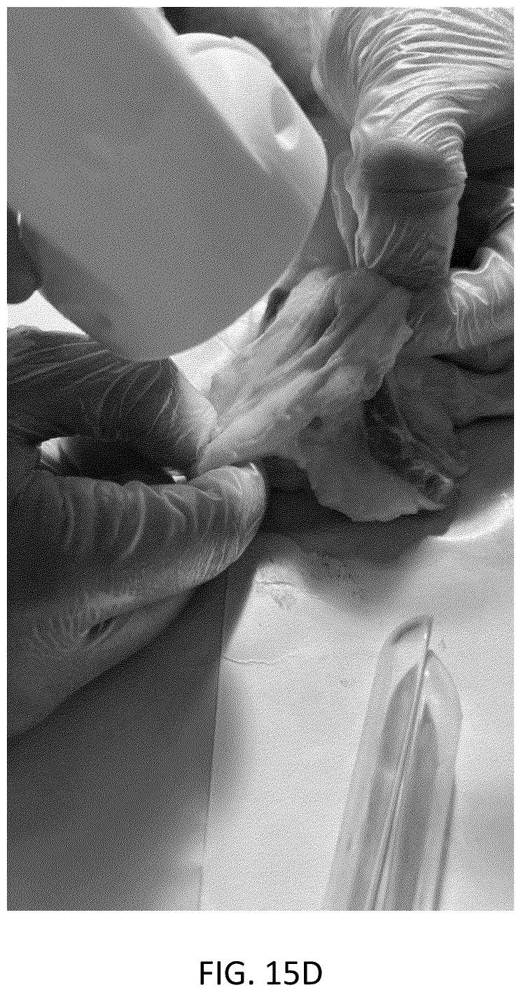

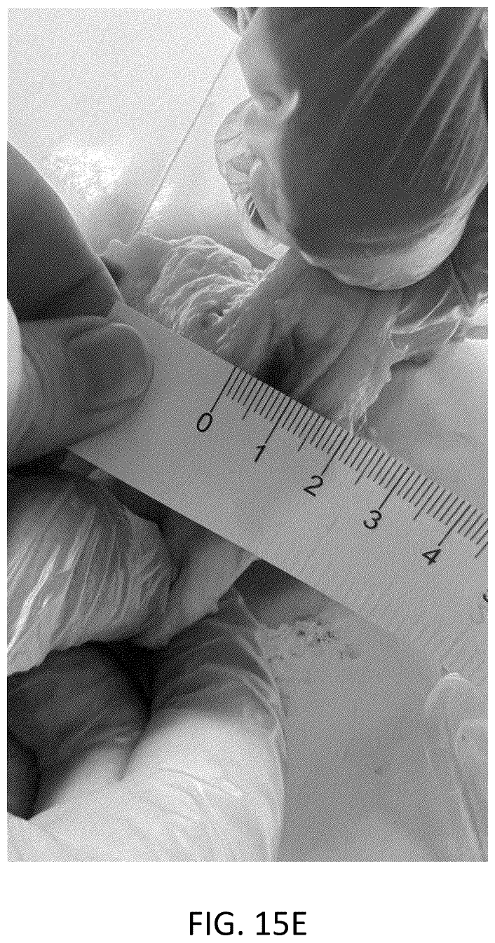

3. Measurement Comparison: Cervical dilation measurements obtained by the exemplary medical device (FIGS. 15A-D) were compared to those taken manually using a medical measuring tape (FIG. 15E). Each measurement was repeated three times to assess repeatability.

Data Analysis: Measurements were analyzed using the following statistical metrics: Mean absolute error (MAE) and mean percentage error (MPE) to assess accuracy; Coefficient of variation (CV) to assess precision; and Bland-Altman analysis to evaluate agreement between the exemplary device and manual measurements.

Results:

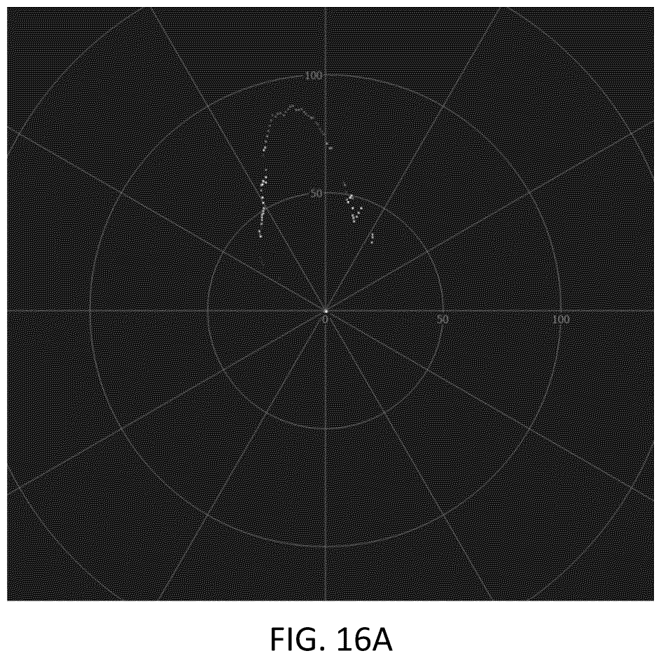

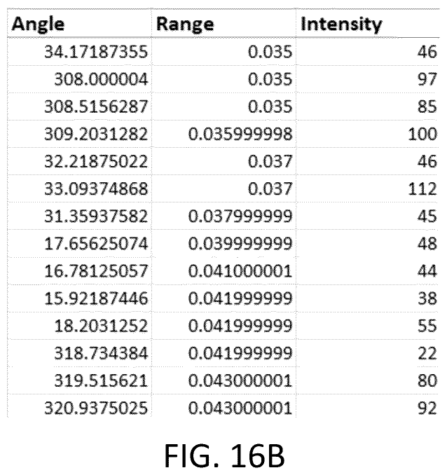

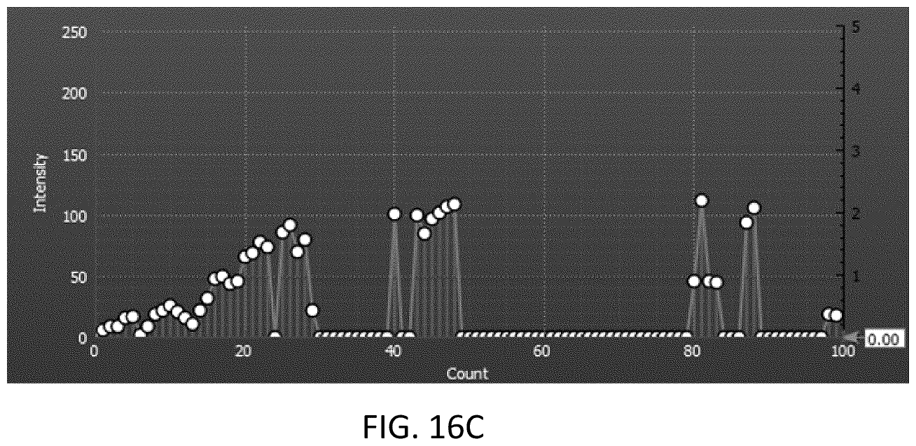

1. High precision mapping: the exemplary device successfully captured detailed point cloud maps of the cervix, enabling precise visualization of cervical anatomy. FIGS. 16A-C provide an example of a point cloud map and associated data generated by the device.

2. Measurement Accuracy: the exemplary device demonstrated high precision in measuring cervical dilation, with minimal differences compared to manual measurements. Table 1 summarizes the measurements obtained by the device and the manual measuring tape, along with the differences between the two methods.

TABLE 1
Comparison of Cervical Dilation Measurements by
the exemplary device and Manual Measuring Tape

| Measurement | Exemplary Device | Measuring Tape | Difference |
|---|---|---|---|
| Number | (mm) | (mm) | (mm) |
| 1 | 38.9 | 35 | 3.86 |
| 2 | 29.8 | 30 | −0.17 |
| 3 | 20.7 | 20 | 0.73 |
| 4 | 15.2 | 15 | 0.19 |
| 5 | 44.5 | 45 | −0.46 |
| 6 | 31.7 | 30 | 1.74 |
| 7 | 24.3 | 25 | −0.70 |
| 8 | 42.2 | 39 | 3.19 |
| 9 | 14.0 | 15 | −0.98 |
| 10 | 46.7 | 45 | 1.72 |
| Mean ± SD | 30.8 ± 11.2 | 29.9 ± 10.5 | 0.9 ± 1.8 |

3. Statistical Analysis:
Mean Absolute Error (MAE): 0.9 mm.
Mean Percentage Error (MPE): 2.1%.
Coefficient of Variation (CV): 3.2%.
Bland-Altman Analysis: The mean difference (bias) was 0.9 mm, with 95% limits of agreement ranging from −2.6 mm to 4.4 mm (FIG. 2).

The results demonstrate that the exemplary device provides accurate and precise measurements of cervical dilation, with a mean absolute error of 0.9 mm and a coefficient of variation of 3.2%. These findings suggest that the exemplary device is a reliable alternative to manual measurements, which are prone to subjectivity and inter-observer variability (Sheiner, E. et al. (2017). Accuracy and consistency of cervical dilation measurements during labor. European Journal of Obstetrics & Gynecology, 215(1), 50-55), herein incorporated by reference.

The Bland-Altman analysis further supports the device's accuracy, showing no significant systematic bias and good agreement with manual measurements.

The high-resolution point cloud maps generated by the exemplary device in FIG. 15 highlight its potential for detailed anatomical visualization, which could aid in monitoring labor progression and identifying abnormalities.

This proof-of-concept study was conducted using non-living sheep reproductive systems and approximates in vivo conditions. Additional studies will evaluate the exemplary device in live animal models and clinical settings to confirm its performance under physiological conditions.

The proof-of-concept studies demonstrate that the exemplary device is a promising tool for accurate and precise measurement of cervical dilation. With a mean absolute error of 0.9 mm and excellent repeatability (CV=3.2%), the exemplary device has the potential to improve obstetric diagnostics and labor management. Additional research will focus on clinical validation and addressing the limitations of this study, including testing in live animal models or human subjects.

### Example 2

### Veterinary care (Horses, Pigs, Other Animals)

The exemplary device is herein contemplated for use in veterinary care. For example, the exemplary device is contemplated for use in both routine care and specialized veterinary procedures for animals including, as non-limiting examples, horses, donkeys, mules, pigs, dogs, cats, pandas, camels, alpacas, sheep, goats and the like. The exemplary device can be used to monitor the progression of pregnancy and labor for any of the aforementioned animals, related animals, and other animals referred to here. The exemplary device can also be used in the examination animals' anatomical structures within their orifices.

Measurements and 2D or 3D spatial mappings of the anatomical structures of veterinary care subjects can be observed.

### Example 3

### Obstetric Care Examinations

Obstetric care patients are individuals can be subjects who are either trying to become pregnant or who are pregnant. Patients who are trying to become pregnant may opt for prenatal pelvic examination to have their anatomical structures assessed in preparation for pregnancy. Patients who are pregnant are examined at several intervals over the course of their pregnancies, and undergo pelvic examinations to track the progress of the developing fetus and the mother's anatomies in preparation for labor.

Measurements and 2D or 3D spatial mappings of the anatomical structures of obstetric care patients can be observed. The measurements and 2D or 3D spatial mappings can be used to provide precise data and information for a given time interval, e.g. for the 18-week check-up. As collected over time, the measurements and spatial mappings can also provide a more complete dataset upon which current and future medical decisions, such as particular interventions including as a non-limiting example, whether a repeat cesarean section or vaginal birth after cesarean section (VBAC) is advisable to the patient.

### Example 4

### Well-Woman Routine Examinations

Female subjects are recommended to begin their routine gynecological examinations between ages 13 and 15. The “well-woman” examination, which includes the pelvic exam, is performed to assess the health of the female organs in the pelvis and includes cervical examination. The well-woman exam is a recommended to be done yearly in order to identify any early signs of disease, cancer, and medical conditions, including pregnancy.

Measurements and 2D or 3D spatial mappings of the anatomical structures of female subjects can be observed and collected over time. It is contemplated that the measurements and spatial mappings are standardizable by using the exemplary medical device.

### Example 5

### Other Anatomies

The exemplary medical device is contemplated for use to measure and spatially map anatomies including, as non-limiting examples, an ear canal, the mouth, nose, anal cavity, and vaginal cavity. The exemplary medical device is also contemplated for use during surgical procedures, including, as a non-limiting example, during a laparoscopic procedure to measure and visualize with 2D or 3D spatial mappings the anatomies within the abdomen or pelvis.

### Example 6

### Examples of the LiDAR-Based Medical Device

Certain configurations of the medical device are contemplated herein.

The medical device can comprise a body coupled to a handle, a tip comprising a plurality of petals defining an opening positioned on a distal end of the body of the medical device, and a LiDAR system comprising a low-level energy laser positioned within the body and proximate the tip wherein the laser is positioned to align with the opening defined by the petals.

The medical device can comprise a body coupled to a handle, a decouplable tip comprising a plurality of petals defining an opening positioned on a distal end of the body of the medical device, and a LiDAR system comprising a low-level energy laser positioned within the body and proximate the tip wherein the laser is positioned to align with the opening defined by the petals.

The medical device can comprise a body comprising: a LiDAR system positioned within the body comprising a low-level energy laser; a speculum coupled to the body, comprising: a conical tip having a plurality of petals defining an opening positioned on a distal end of the body of the medical device; and an adjustment element that controls a size of the opening by movement of at least some of the plurality of the petals during use; wherein the laser is fixedly positioned within the body to align with the opening defined by the petals. The body can be rigid, in that it will not bend after insertion into the intrusui of the subject. A rigid body ensures that the scanned region of the anatomy is the selected region, because movement of the subject, or scanned region (e.g., cervix) can otherwise devoid the accuracy of the scanning process.

The tip of the medical device can function as a speculum or dilator. The tip of the medical device comprises petals which define an opening positioned on a distal end of the body of the device. The tip can function as a dilator by the ability of each petal of an exemplary tip (102) to rotate about a hinge in response to an inner dilating component (103) being rotated as controlled by an adjusting knob (104). Rotation in one direction of the adjusting knob (104) controlling the rotation of an inner dilating component (103), e.g., clockwise, can result in the controlled dilation of an exemplary tip. Conversely, a counter-directional rotation of the adjusting knob (104) controlling the rotation of an inner dilating component (103), e.g., counterclockwise, can result in the controlled closure of an exemplary tip. Accordingly, the tip can have a closed configuration and an open configuration with a range of diameters, up to and including about 9 cm. The tip can be coated or not coated. The tip of the medical device can comprise at least two petals. The tip of the medical device can comprise at least three petals. The tip of the medical device can comprise at least four petals. The tip of the medical device can comprise at least five petals. The tip of the medical device can comprise more than five petals.

In some embodiments, this disclosure provides for the following numbered embodiments:
A1. A medical device comprising: a body coupled to a handle; a tip comprising a plurality of petals defining an opening positioned on a distal end of the body of the medical device; and a light detection and ranging (LiDAR) system comprising a low-level energy laser positioned within the body and proximate the tip; wherein the laser is positioned to align with the opening defined by the petals.
A2. The medical device of paragraph A1, wherein the opening has a diameter in a range from about 0.75 cm to about 9 cm.
A3. The medical device of paragraph Al further comprising an adjustment element that controls movement of at least some of the plurality of the petals during use.
A4. The medical device of paragraph A1 further comprising an adjustment element that controls movement of at least some of the plurality of the petals such that a user determines a position of the petals during use.
A5. The medical device of paragraph Al further comprising an adjustment element that controls movement of at least some of the plurality of the petals such that the opening diameter is in a range from 1 cm to 7 cm during use.
A6. The medical device of paragraph A1, wherein the laser is fixedly positioned proximate the opening in the tip during use.
A7. The medical device of paragraph A1, wherein the LiDAR system comprises at least one of static LiDAR, near-field LiDAR, micropulse LiDAR, photon counting LiDAR, hybrid solid-state type LiDAR, flooded light array type (FLASH) LiDAR, time-of-flight (ToF) LiDAR, scanning LiDAR, solid-state LiDAR, or a sequence thereof, or combination thereof.
A8. The medical device of paragraph A1, wherein the low-energy laser emits light at a wavelength in a range from 400 nanometers to 1000 nanometers.
A9. The medical device of paragraph A1, wherein the plurality of petals comprises at least three petals.
A10. The medical device of paragraph A1, wherein the plurality of petals comprises at least five petals.
A11. The medical device of paragraph Al wherein the plurality of petals comprises at least one of polyurethane, polyphenylsulfone, heat-stabilized polypropylene, polyetheretherketone, polyvinyl chloride, acetal copolymer, ultra-high molecular weight polyethylene, or a combination thereof.
A12. The medical device of paragraph A1, further comprising a processor and a display screen.
A13. A medical device comprising: a body comprising: a light detection and ranging (LiDAR) system positioned within the body comprising a low-level energy laser; a speculum coupled to the body, comprising: a conical tip having a plurality of petals defining an opening positioned on a distal end of the body of the medical device; and an adjustment element that controls a size of the opening by movement of at least some of the plurality of the petals during use; wherein the laser is fixedly positioned within the body to align with the opening defined by the petals.
A14. The medical device of paragraph A13 wherein a diameter of the opening is in a range from 1 cm to 7 cm during use.
A15. The medical device of paragraph A13 wherein the plurality of petals are arranged such that the conical tip is in an insertion configuration when the diameter of the opening is less than about 2 cm.
A16. A method for providing a spatial mapping of an anatomy comprising: providing a medical device comprising: a speculum comprising: a plurality of polymer petals; a light detection and ranging (LiDAR) system comprising: a low-level energy laser emitting electromagnetic radiation comprising at least infrared radiation, visible radiation, invisible infrared radiation, near infrared radiation, or a combination thereof; inserting the speculum into an orifice of a subject; and using the speculum to dilate the orifice of the subject; using the LiDAR system to generate the spatial mapping of an anatomy; capturing the spatial mapping; analyzing the spatial mapping; and displaying the spatial mapping.
A17. The method of paragraph A16, further comprising: identifying at least a feature of an anatomy comprising a measurement, position, consistency, vascularization, effacement, tissue softening, fetal station, fetal measurement, or a combination thereof; and capturing, analyzing, and displaying the feature.
A18. The method of paragraph A16, further comprising: identifying a cervical effacement and tissue softness, capturing features of cervical effacement and tissue softness, and analyzing and displaying the features of cervical effacement and tissue softness.
A19. The method of paragraph A16, further comprising: identifying a region of rigid or damaged tissue, capturing features of the region of rigid or damaged tissue, and analyzing and displaying the features of the region of rigid or damaged tissue.
A20. The method of paragraph A16 wherein the speculum has an opening defined by the petals having a diameter of less than 2 cm during insertion; and the opening has a diameter in a range from about 2 cm to about 7 cm during dilation of the orifice of the subject.

Having thus described in detail preferred embodiments, it is to be understood that the invention defined by the above paragraphs is not to be limited to particular details set forth in the above description as many apparent variations thereof are possible without departing from the spirit or scope of the present invention.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents. Reference to any applications, patents and publications in this specification is not, and may not be taken as, an acknowledgment or any form of suggestion that they constitute valid prior art or form part of the common general knowledge in any country in the world.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or a negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed is:

1. A medical device comprising:
a body coupled to a handle;
a tip comprising a plurality of petals defining an opening positioned on a distal end of the body of the medical device; and
a light detection and ranging (LiDAR) system comprising a low-level energy laser positioned within the body and proximate the tip;
wherein the laser is positioned to align with the opening defined by the petals.

2. The medical device of claim 1, wherein the opening has a diameter in a range from about 0.75 cm to about 9 cm.

3. The medical device of claim 1 further comprising an adjustment element that controls movement of at least some of the plurality of the petals during use.

4. The medical device of claim 1, wherein the body is rigid.

5. The medical device of claim 1 further comprising an adjustment element that controls movement of at least some of the plurality of the petals such that the opening diameter is in a range from 1 cm to 7 cm during use.

6. The medical device of claim 1, wherein the laser is fixedly positioned proximate the opening in the tip during use.

7. The medical device of claim 1, wherein the LiDAR system comprises at least one of static LiDAR, near-field LiDAR, micropulse LiDAR, photon counting LiDAR, hybrid solid-state type LiDAR, flooded light array type (FLASH) LiDAR, time-of-flight (ToF) LiDAR, scanning LiDAR, solid-state LiDAR, or a sequence thereof, or combination thereof.

8. The medical device of claim 1, wherein the low-energy laser emits light at a wavelength in a range from 400 nanometers to 1000 nanometers.

9. The medical device of claim 1, wherein the plurality of petals comprises at least three petals.

10. The medical device of claim 1, wherein the plurality of petals comprises at least five petals.

11. The medical device of claim 1 wherein the plurality of petals comprises at least one of polyurethane, polyphenylsulfone, polypropylene, polyetheretherketone (PEEK), polyvinyl chloride, acetal copolymer, ultra-high molecular weight polyethylene, or a combination thereof.

12. The medical device of claim 1, further comprising a processor and a display screen.

13. A medical device comprising:
a body comprising:
a light detection and ranging (LiDAR) system positioned within the body comprising a low-level energy laser;
a speculum coupled to the body, comprising:
a conical tip having a plurality of petals defining an opening positioned on a distal end of the body of the medical device; and
an adjustment element that controls a size of the opening by movement of at least some of the plurality of the petals during use;
wherein the laser is fixedly positioned within the body to align with the opening defined by the petals.

14. The medical device of claim 13 wherein a diameter of the opening is in a range from 1 cm to 7 cm during use.

15. The medical device of claim 13 wherein the plurality of petals are arranged such that the conical tip is in an insertion configuration when the diameter of the opening is less than about 2 cm.

16. A method for providing a spatial mapping of an anatomy comprising:
providing a medical device comprising:
a speculum comprising:
a plurality of polymer petals;
a light detection and ranging (LiDAR) system comprising:
a low-level energy laser emitting electromagnetic radiation comprising at least infrared radiation, visible radiation, invisible infrared radiation, near infrared radiation, or a combination thereof;
inserting the speculum into an orifice of a subject; and
using the speculum to dilate the orifice of the subject;
using the LiDAR system to generate the spatial mapping of an anatomy;
capturing the spatial mapping;
analyzing the spatial mapping; and
displaying the spatial mapping.

17. The method of claim 16, further comprising:
identifying at least a feature of an anatomy comprising a measurement, position, consistency, vascularization, effacement, tissue softening, fetal station, fetal measurement, or a combination thereof; and
capturing, analyzing, and displaying the feature.

18. The method of claim 16, further comprising:
identifying a cervical effacement and tissue softness,
capturing features of cervical effacement and tissue softness, and
analyzing and displaying the features of cervical effacement and tissue softness.

19. The method of claim 16, further comprising:
identifying a region of rigid or damaged tissue,
capturing features of the region of rigid or damaged tissue, and
analyzing and displaying the features of the region of rigid or damaged tissue.

20. The method of claim 16 wherein the speculum has an opening defined by the petals having a diameter of less than 2 cm during insertion; and the opening has a diameter in a range from about 2 cm to about 7 cm during dilation of the orifice of the subject.
